(19) United States
(12) Patent Application Publication
(10) Pub. No.: US 2019/0042702 A1
(43) Pub. Date: Feb. 7, 2019

(54) COGNITIVE MEDICATION CONTAINERS PROVIDING TARGETED MEDICATION ASSISTANCE

(71) Applicant: INTERNATIONAL BUSINESS MACHINES CORPORATION, ARMONK, NY (US)

(72) Inventors: Varun Chandramouli, Cambridge, MA (US); Anca Sailer, Scarsdale, NY (US); Sanjay Surendranath Girija, Buffalo, NY (US); Mahesh Viswanathan, Yorktown Heights, NY (US)

(21) Appl. No.: 15/667,675

(22) Filed: Aug. 3, 2017

(51) Int. Cl. G06F 19/00; A61B 5/00

(52) U.S. Cl. CPC G06F 19/3481; A61B 5/4839; G06F 19/00; G06F 19/3456; G06F 19/324

(57) ABSTRACT

The program directs a computer processor to implement a program that manages a device. The program stores a medication listing, together with medication consumption instructions. The program monitors one or more medication containers, which contain at least one medication for the patient. The program receives medication consumption data of the patient, including the name of the drug consumed, the quantity, and the time that the patient last consumed the one or more medications. The program receives patient condition data from one or more monitoring devices in real-time, which includes at least one measurement of a medical vital sign of the patient. Based on the patient condition data, the program recommends that the patient consume a dosage amount of a medication at a designated time, from the one or more medication containers, or locks the one or more medication containers to prevent an adverse drug reaction or overdose in the patient.

### BACKGROUND

The present disclosure relates generally to patient health and safety with reference to consuming multiple prescription drugs from various medication containers, and more particularly to safeguarding a patient's health by cognitively predicting adverse drug combinations, and preventing access to drugs contained within various medication containers, based on predicted adverse drug combinations.

An adverse drug reaction is an injury caused by taking a medication, and may occur following a single dose or a prolonged administration of a drug or as a result from the combination of two or more drugs.

Oftentimes, adverse drug reactions may occur due to patients having multiple physicians who each prescribe multiple drugs without knowing the other drugs prescribed by the other physicians which may result in a patient consuming a dangerous drug combination that may result in a hospitalization or even death.

In other scenarios, the patient may be to blame for an adverse drug reaction. The patient may mistakenly consume multiple doses of a medication when they are only supposed to consume one dose, or mistakenly take a medication from the incorrect medication container, or even consume a medication when their vital signs are not an optimum level to receive those medications. Failing to provide a correct dosing of medications may lead to adverse drug reactions in the patient.

### BRIEF SUMMARY

Embodiments of the present invention disclose a method, a computer program product, and a system.

According to an embodiment, a method, in a data processing system including a processor and a memory, for implementing a program that manages a device. The method stores a medication listing for a patient, wherein the medication listing lists at least one medication for the patient, together with medication consumption instructions. The method monitors one or more medication containers, wherein the one or more medication containers contain at least one medication for the patient. The method further receives, from the one or more medication containers, medication consumption data of the patient, wherein medication consumption data includes which medication of the at least one medication the patient has taken, a time that the patient took the at least one medication, and a quantity of the at least one medication taken by the patient. The method also receives patient condition data from one or more patient monitoring devices in real-time, wherein the patient condition data includes at least one measurement of a medical vital sign of the patient. The method recommends that the patient consume a dosage amount of a medication at a designated time, from the one or more medication containers, based on the patient condition data.

According to another embodiment, a computer program product for directing a computer processor to implement a program that manages a device. The storage device embodies program code that is executable by a processor of a computer to perform a method. The method stores a medication listing for a patient, wherein the medication listing lists at least one medication for the patient, together with medication consumption instructions. The method monitors one or more medication containers, wherein the one or more medication containers contain at least one medication for the patient. The method further receives, from the one or more medication containers, medication consumption data of the patient, wherein medication consumption data includes which medication of the at least one medication the patient has taken, a time that the patient took the at least one medication, and a quantity of the at least one medication taken by the patient. The method also receives patient condition data from one or more patient monitoring devices in real-time, wherein the patient condition data includes at least one measurement of a medical vital sign of the patient. The method recommends that the patient consume a dosage amount of a medication at a designated time, from the one or more medication containers, based on the patient condition data.

According to another embodiment, a system for implementing a program that manages a device, includes one or more computer devices each having one or more processors and one or more tangible storage devices. The one or more storage devices embody a program. The program has a set of program instructions for execution by the one or more processors. The program instructions include instructions for storing a medication listing for a patient, wherein the medication listing lists at least one medication for the patient, together with medication consumption instructions. The program instructions include instructions for monitoring one or more medication containers, wherein the one or more medication containers contain at least one medication for the patient. The program instructions include instructions for receiving, from the one or more medication containers, medication consumption data of the patient, wherein medication consumption data includes which medication of the at least one medication the patient has taken, a time that the patient took the at least one medication, and a quantity of the at least one medication taken by the patient. The program instructions include instructions for receiving patient condition data from one or more patient monitoring devices in real-time, wherein the patient condition data includes at least one measurement of a medical vital sign of the patient. The program instructions include instructions for recommending that the patient consume a dosage amount of a medication at a designated time, from the one or more medication containers, based on the patient condition data.

### BRIEF DESCRIPTION OF THE DRAWINGS

FIG. 1 illustrates a computing environment, in accordance with an embodiment of the present invention.

FIG. 2 is a block diagram illustrating the features of controlled action generator 128 of FIG. 1, in accordance with embodiments of the present invention.

FIG. 3 illustrates the storage of data objects within medication consumption database 144 of FIG. 1, in accordance with embodiments of the present invention.

FIG. 4 is a diagram illustrating the peer to peer network communication of medication containers 130 of FIG. 1, in accordance with embodiments of the present invention.

FIG. 5 is a flowchart illustrating the operation of medication assistant controller 118 of FIG. 1, in accordance with an embodiment of the present invention.

FIG. 6 is a diagram graphically illustrating the hardware components of a computing environment of FIG. 1, in accordance with an embodiment of the present invention.

FIG. 7 depicts a cloud computing environment, in accordance with an embodiment of the present invention.

FIG. 8 depicts abstraction model layers of the illustrative cloud computing environment of FIG. 7, in accordance with an embodiment of the present invention.

### DETAILED DESCRIPTION

The present invention discloses a method which includes one or more medical containers (e.g. bottle, jar, medication organizer, can, piston, syringe etc.) for a pharmacist, doctor, patient, nurse or medical assistant to store individual medications or drugs, oftentimes prescription drugs that may contain instructions for use, and a means for estimating a cognitive feature of a user of the one or more medical containers. A cognitive feature may include detecting outside factors such as the vital signs of a user, adverse drug to drug interactions between scheduled dosages of one or more different drugs, and adverse drug to drug interactions that may have occurred within a user's body based on previously consumed drugs. Based on the one or more medical containers use and the additional cognitive feature of the invention, the system may take a controlled action that benefits the user. Drug to drug interactions may be evaluated by querying a cloud service providing APIs to this aim.

It is important to note that not only prescription drugs for a user may be detected by the current system. For example, over the counter drugs may be stored in the one or more medication containers. In this scenario, the system may receive a user inputting the name of the drug, and dosage, into the system in order to adequately search for adverse drug to drug interactions. Notwithstanding the above, a patient's vital signs may be detected to alert the system that a user is experiencing adverse medical reactions or even an overdose. In response, a cognitive feature of the invention may lock the one or more medication containers, and notify emergency services, to prevent the user from consuming additional drugs.

The method may, optionally, detect travel plans for a user based on a feed from a user's electronic calendar and notify the user if a replenishment of a drug is required prior to going away on an extended stay away from home.

Existing medication containers lack the cognitive feature to safeguard a patient from inadvertent adverse drug reactions. The present invention includes a smart technology medication container (i.e. one that may detect potential adverse reactions or actual adverse reactions between drug combinations) linked to a centralized software application which may be helpful in protecting a user's medical health and minimizing the number of adverse drug reactions, and resulting visits to a doctor's office and/or hospital.

Hereinafter, exemplary embodiments of the present invention will be described in detail with reference to the attached drawings.

The present invention is not limited to the exemplary embodiments below, but may be implemented with various modifications within the scope of the present invention. In addition, the drawings used herein are for purposes of illustration, and may not show actual dimensions.

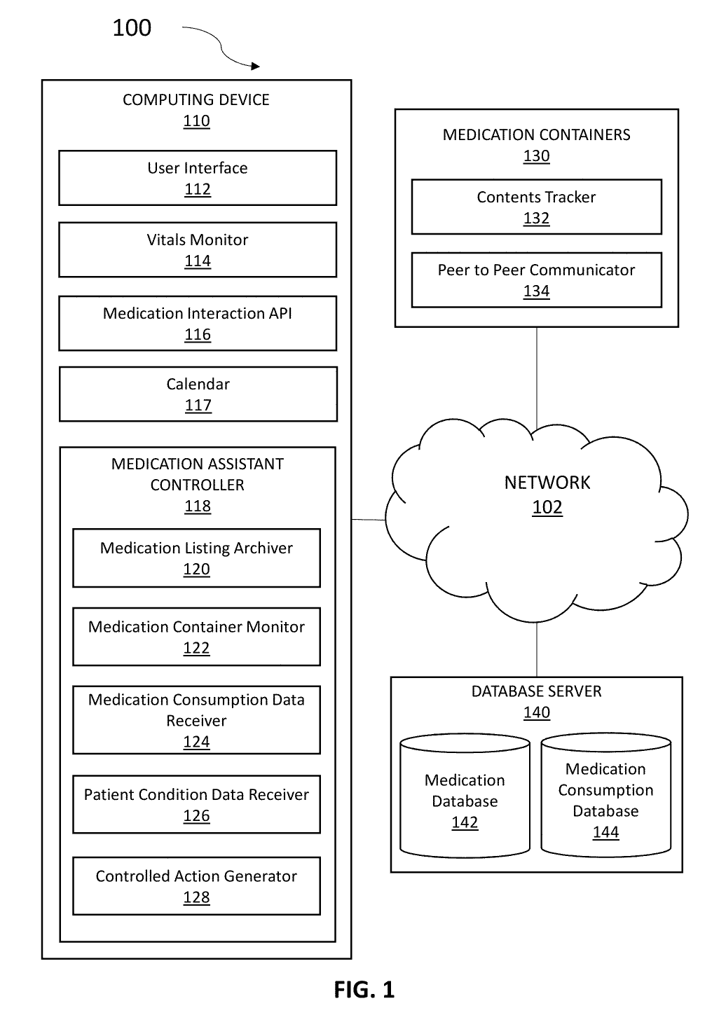

FIG. 1 is a functional block diagram of a computing environment 100, according to an embodiment of the present invention. Computing environment 100 includes computing device 110, medication containers 130, and database server 140 all connected via network 102. The setup in FIG. 1 represents an example embodiment configuration for the present invention, and is not limited to the depicted setup in order to derive benefit from the present invention. Furthermore, the term “medication” may be used interchangeably with the term “drug” throughout the application.

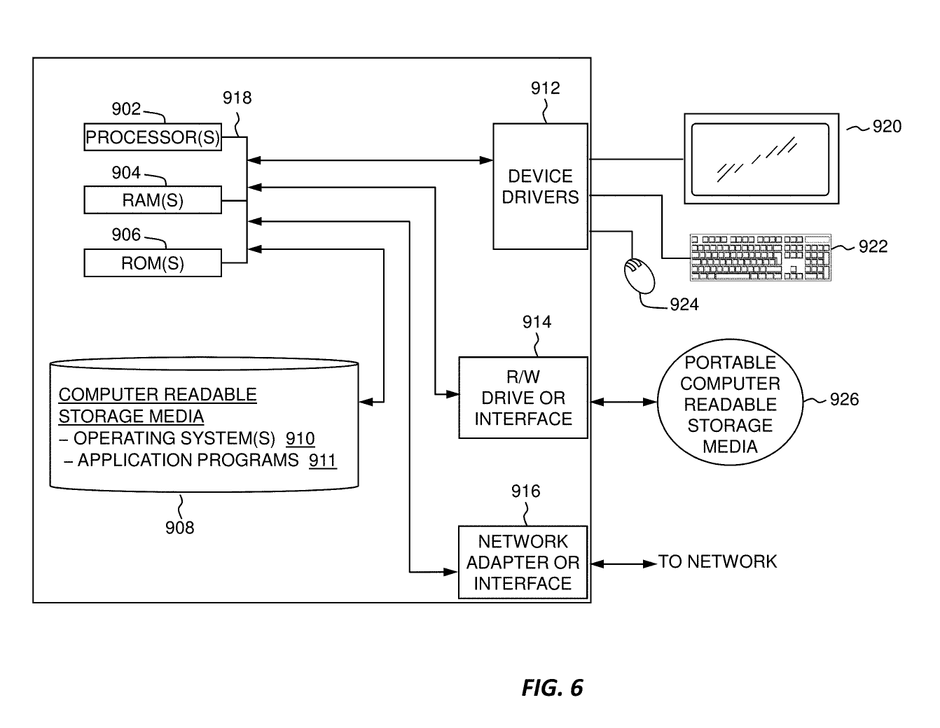

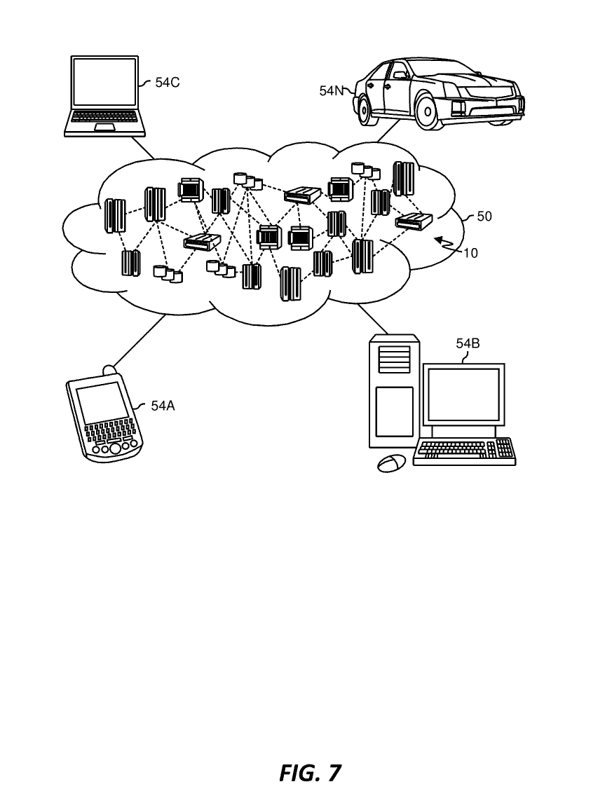

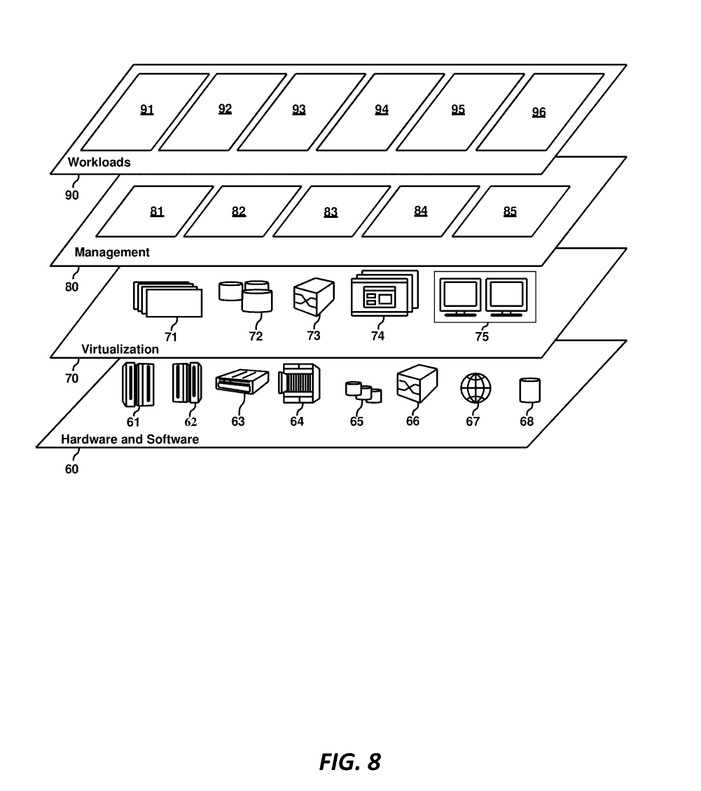

In the example embodiment, computing device 110 contains user interface 112, vitals monitor 114, medication interaction application programming interface (API) 116, calendar 117 and medication assistant controller 118. In various embodiments, computing device 110 may be a laptop computer, tablet computer, netbook computer, personal computer (PC), a desktop computer, a personal digital assistant (PDA), a smart phone, or any programmable electronic device capable of communicating with medication containers 130 and database server 140 via network 102. Computing device 110 may include internal and external hardware components, as depicted and described in further detail below with reference to FIG. 6. In other embodiments, computing device 110 may be implemented in a cloud computing environment, as described in relation to FIGS. 7 and 8, herein. Computing device 110 may also have wireless connectivity capabilities allowing it to communicate with medication containers 130, database server 140, and other computers or servers over network 102.

In the example embodiment, computing device 110 includes user interface 112, which may be a computer program that allows a user to interact with computing device 110 and other connected devices via network 102. For example, user interface 112 may be a graphical user interface (GUI). In addition to comprising a computer program, user interface 112 may be connectively coupled to hardware components, such as those depicted in FIG. 6, for receiving user input. In the example embodiment, user interface 112 is a web browser, however in other embodiments user interface 112 may be a different program capable of receiving user interaction and communicating with other devices.

In the example embodiment, vitals monitor 114 may be a computer program, on computing device 110, that detects and monitors a user's vital signs which may include blood pressure, cholesterol levels, blood sugar levels, heart rate and so on. In other embodiments, vitals monitor 114 may be a separate device such as a blood glucose monitor, a heart rate monitor, or a wearable device that detects one or more of a user's vital signs, and communicates with computing device 110. Vitals monitor 114 outputs detected and monitored vital signs of a user to medication assistant controller 118, either on a continuous basis or at set intervals. In other embodiments, vitals monitor 114 may be configured to detect and monitor a user's vital signs based on any method known in the art.

In the example embodiment, medication interaction API 116 may be a cloud based API that monitors medication reactions based on a quantity (Q), or dosage amount, and a time of consumption (T) based on various rules. Medication interaction API 116, in the example embodiment, receives medication information of a user from medication assistant controller 118, and returns a recommended list of medications based on a particular user's physical profile (e.g. height, weight, age), medical profile (e.g. allergies, medical conditions), and vital signs. Medication interaction API 116, in the example embodiment, may receive a user's medication list and associated medical prescriptions from medication database 142, and a user's medication consumption data from medication consumption database 144.

For example, medication interaction API 116 may receive a user's heart rate information, together with the current medications that a user is metabolizing. Based on this information, medication interaction API 116 is capable of detecting adverse reactions of multiple combinations of medications, both currently in the user's body, as well as future scheduled dosages pursuant to a user's medication prescriptions. Medication interaction API 116 is therefore capable of detecting overdoses and/or adverse reactions of medication combinations either before they take place or while they are taking place.

In an example embodiment, medication interaction API 116 is further capable of recommending a dosage amount of a medication to a user to alleviate one or more medical conditions that a user may currently be experiencing. The recommended dosage amount of a medication may either be immediately available to user (i.e. contained within a user's prescription regimen) or it may be a medication that is not currently in the user's possession. In the latter scenario, medication interaction API 116 may be capable of sending the prescription information directly to the user's physician's office or pharmacy for confirmation and/or fulfillment. In the example embodiment, medication interaction API 116 evaluates adverse medication interactions between any proposed medications, to a user, and any prescribed medications that a user has currently taken (i.e. is currently metabolizing, as well as the length of time left for a medication to fully metabolize within the user's body), or is scheduled to take in the future.

In the example embodiment, calendar 117 may be a computer program, on computing device 110, that syncs a user's electronic calendar from another computing device or application to calendar 117. Calendar 117 may include a user's personal calendar such as birthdays, vacation dates, travelling schedule, personal event information, as well as a user's work calendar such as meetings, conference dates, travelling schedule, and so forth. Calendar 117, in the example embodiment, is capable of communicating with medication assistant controller 118, and as such, receives access to medication database 142 and medication consumption database 144. Calendar 117 is further capable of detecting whether a user has enough supply of a medication based on future calendar events where the user may be out of town and unable to re-supply a particular medication.

In the example embodiment, database server 140 includes medication database 142 and medication consumption database 144, and may be a laptop computer, tablet computer, netbook computer, personal computer (PC), a desktop computer, a personal digital assistant (PDA), a smart phone, a server, or any programmable electronic device capable of communicating with computing device 110 and medication containers 130 via network 102. While database server 140 is shown as a single device, in other embodiments, database server 140 may be comprised of a cluster or plurality of computing devices, working together or working separately.

In the example embodiment, medication database 142 contains a list of one or more medications, or prescribed drugs, for a user, and stored as a data object in the following format: <drugName, dosage, instructions>. In other embodiments, a list of prescriptions may be stored in medication database 142 in any fashion known to one of ordinary skill in the art. In alternative embodiments, a user's emergency medical record (EMR) or medical file at the doctor's office may be linked to medication database 142. In other embodiments, medication database 142 may also be linked to the user's pharmacy, where a pharmacist may verify a prescription directly with the user's prescribing physician. Instructions for a prescribed drug may include “take twice daily”, “take with water”, “take with food”, “take once every four hours”, and so on. In the exemplary embodiment, prescribed drugs may be added or removed from medication database 142 by the user's prescribing physician. Another source for populating medication database 142 may be pinging the user's medication containers, which contain a control unit capable of detecting the drug within the medication container, together with the quantity and instructions for use. Additionally, medication database 142 may store lists of one or more medications associated with various users based on the control unit, within each medication container, identifying with a particular user and a particular network of medication containers for the user. In alternative embodiments, medication database 142 may filter a user's lists of medications according to prescribed drugs, over-the-counter drugs, and so forth.

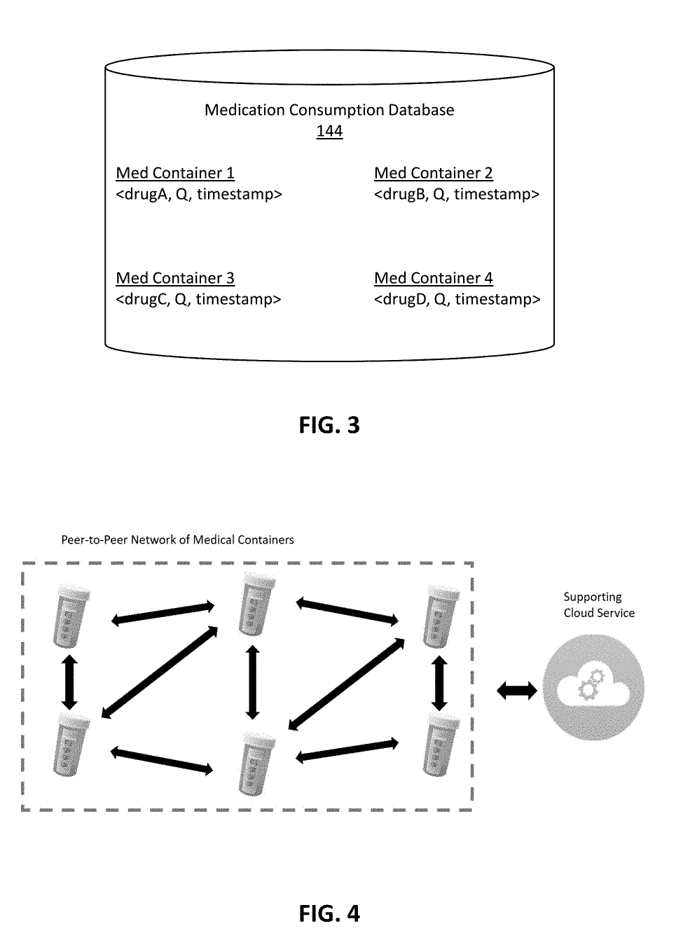

In the example embodiment, and with reference to FIG. 3, medication consumption database 144 contains information detailing a user's consumption, or intake, of a particular medication listed in medication database 142. Medication consumption database 144 receives data input from one or more medication containers 130, which include one or more medications for a user. In the exemplary embodiment, medication consumption database 144 denotes which medication, or drug, a user consumes, the quantity consumed (Q), and the time that the user consumed it. This information may be stored as a data object within medication consumption database 144, as depicted in FIG. 3, as follows: <drugName, Q, timestamp>. In alternative embodiments, medication consumption database 144 may be stored within the software component of one or more medication containers 130.

In the exemplary embodiment, medication consumption database 144 communicates with medication containers 130 and medication assistant controller 118. Each medication container houses a particular drug with a specific prescription instruction for consuming that particular drug. The medication containers have a unique ability to detect which drug is being consumed, in what dosage, and at what time, as will be discussed below with reference to medication containers 130. Medication consumption database 144 keeps track of a user's drug consumption throughout a twenty-four-hour cycle to help prevent a consumption of multiple drugs, within a certain timeframe, that may cause adverse reactions within the user if combined. In the exemplary embodiment, the data object <drugA, 50 mg, 9:00 am> may be automatically deleted from medication consumption database 144 at 1:00 pm since drugA is fully metabolized within the human body after four hours, and therefore no longer a risk for contributing to a combination of medications that may cause an adverse reaction. In other embodiments, the data object <drugA, 50 mg, 9:00 am> may be saved for record keeping purposes, even though it is no longer applicable in helping to prevent a consumption of multiple drugs that may cause adverse reactions.

In the example embodiment, medication containers 130 may be one or more bottles, jars, medication organizers, syringes, or any other medication container, having one or more electronic control units (having a processor, a tangible memory, and program code stored on the tangible memory for execution by the processor) that includes a contents tracker 132, and a peer to peer communicator 134, capable of communicating with computing device 110 and database server 140 via network 102. While medication containers 130 is shown as a cluster or plurality of computing devices working together, in other embodiments, medication containers 130 may be comprised of a single device working separately.

Contents tracker 132, in the exemplary embodiment, may be a programmable logic component capable of detecting the contents of individual medication containers 130, as well as timestamping the removal of its contents. In the exemplary embodiment, the programmable logic component contained within medication containers 130 may include visual recognition and/or weight detection technology to track when a user takes a particular dose of a medication to identify the quantity and particular medication being consumed. In alternative embodiments, the programmable logic component may include other features, known to one of ordinary skill in the art, that are capable of identifying and measuring the contents of a container and detecting when and how much of the contents are removed by a user. Additionally, contents tracker 132 may be capable of denoting the date and time (i.e. timestamping) when a quantity of a medication is detected as being removed from a particular medication container 130. In the exemplary embodiment, the denoted date and time, together with the medication name and dosage amount, may be stored in medication consumption database 144.

Medication containers 130, in the exemplary embodiment, may include a cap or a lid, containing a smart locking component capable of preventing, or blocking, a user or someone other than a user, from opening the medication containers 130. The smart locking component may also include a control unit capable of automatically locking the cap or lid. For example, medication containers 130, in the exemplary embodiment, may detect a user's unique fingerprint on the outside of each individual medication containers 130 by means of fingerprint identification technology, known to one of ordinary skill in the art. In alternative embodiments, medication containers 130 may identify a user by visual recognition technology (e.g. facial recognition), or by any other means known to one of ordinary skill in the art.

With reference to FIG. 4, medication containers 130 may include a cluster, or plurality, of multiple containers 130 communicating together, via a peer to peer communicator 134, over a peer to peer network (P2P network). A P2P network may be a distributed application architecture that partitions tasks or workloads between peers, or nodes. An individual peer, or node, in the exemplary embodiment, may be one medication containers 130, while a P2P network may include multiple, interconnected, medication containers 130 that share resources amongst each other without the use of a centralized administrative system. For example, each medication containers 130 may be capable of communicating, via peer to peer communicator 134, with any other medication containers 130 within the P2P network. In the exemplary embodiment, information that may be transmitted between the medication containers 130 in the P2P network includes medication consumption data from medication consumption database 144, a user's prescription, or any other drug, data (from medication database 142), and adverse reaction data (as determined by medication interaction API 116).

Peer to peer communicator 134, in the exemplary embodiment, may be Bluetooth or WiFi technology that enables medication containers 130 to be cognitive of the contents of, and user interactions with, the one or more medication containers 130 within the plurality of medication containers 130. For example, a user may have a list of three (3) medications prescribed by a physician, all three medications being contained in separate medication containers 130 (e.g. Medication Container #1<drugA>, Medication Container #2<drugB>, Medication Container #3<drugC>). In the exemplary embodiment, medication containers 130 may each contain a label on the outside of each medication containers 130 identifying the medication name, dosage amount, and consumption instructions (e.g. <drugA, 30 mg, twice daily>, <drugB, 50 mg, take once w food>, <drugC, 100 mg, every four hours>). The medication containers' 130 electronic control unit may contain the same identifying information, in electronic form, as the label on the outside of the individual medication containers 130. The electronic control unit associated with the individual medication containers 130, in the exemplary embodiment, may be capable of communicating the medication name, dosage amount, and consumption instructions with the other medication containers 130 within the P2P network, together with the actual consumption amount and time of consumption, by the user, via peer to peer communicator 134. In alternative embodiments, the electronic control unit associated with the individual medication containers 130 may be capable of communicating the medication name, dosage amount, and consumption instructions, together with the actual consumption amount and time of consumption, by the user, with the centralized application (medication assistant controller 118).

In the exemplary embodiment, peer to peer communicator 134 may enlist additional medication containers 130 to an existing P2P network by verifying the user's unique fingerprint associated with the new mediation containers 130 as the signature for entry. When user-verified, new medication containers 130 come into the proximity of the existing medication containers 130 in the P2P network, the new medication containers 130 announce themselves with a tag, which is then added to the list of peers by the existing medication containers 130, who then broadcast this list until all of the medication containers 130 are queried. Similarly, content information of the various medication containers 130 are conveyed throughout the P2P network when a user picks up (i.e. fingerprint acknowledged) a medication container 130 and is about to consume a dosage of the medication: a chain of messages may be broadcast to all of the other medication containers 130 in the list of peers to notify them of the current medication about to be consumed by the user. This information may then be used by the other medication containers 130, via medication interaction API 116, to determine potential adverse drug reactions if the user actually consumes the medication. Based on the medication interaction API 116 results, the about to be consumed medication container 130 may be automatically locked, via the smart technology within the cap or lid, thus preventing the user from consuming the medication, if determined that the about to be consumed dosage of a medication may adversely react with other medications that are currently in the user's body, or scheduled to be in the user's body. Alternatively, the cap or lid remains unlocked and the user may be permitted to consume the medication.

With continued reference to FIG. 1, medication assistant controller 118, in the example embodiment, may be a computer application on computing device 110 that contains instruction sets, executable by a processor. The instruction sets may be described using a set of functional modules. Medication assistant controller 118 receives input from vitals monitor 114, medication interaction API 116, calendar 117, medication containers 130, and medication database 142. Medication assistant controller 118 outputs data based on various conditions, such as: notifications to refill a medication container if it is detected to be empty or near empty; alerts to take a particular medication to bring vitals to normal; reminders to take a medication based on missed scheduled dosages; and instructions to lock a medication container and notify emergency services in the event of potential adverse drug combinations. In alternative embodiments, medication assistant controller 118 may be a standalone program on a separate electronic device.

Medication assistant controller 118 may be configured to store a medication listing for a user, together with medication consumption instructions, and monitor one or more medication containers, wherein the one or more medication containers contain at least one medication for the patient. Medication assistant controller 118 receives, from the one or more medication containers, medication consumption data of the patient, wherein medication consumption data includes which medication of the at least one medication the patient has taken, a time that the patient took the at least one medication, and a quantity of the at least one medication taken by the patient. Medication assistant controller 118 also receives patient condition data from one or more patient monitoring devices, in real-time, wherein the patient condition data includes at least one measurement of a medical vital sign of the patient. Based on the received medication consumption data and patient condition data, medication assistant controller 118 recommends that the patient consume a dosage amount of a medication at a designated time, from the one or more medication containers.

With continued reference to FIG. 1, the functional modules of medication assistant controller 118 include medication listing archiver 120, medication container monitor 122, medication consumption data receiver 124, patient condition data receiver 126, and controlled action generator 128.

Medication listing archiver 120 includes a set of programming instructions, in medication assistant controller 118, to store a medication listing for a patient, wherein the medication listing lists at least one medication for the patient, together with medication consumption instructions. The set of programming instructions is executable by a processor. In the exemplary embodiment, medication listing archiver 120 contains program instructions to receive and store one or more medications, or prescriptions, from a user's physician in medication database 142. In an exemplary embodiment, only a user's physician is capable of adding or removing prescriptions from the medication database 142.

Medication container monitor 122 includes a set of programming instructions in medication assistant controller 118, to monitor one or more medication containers 130, wherein the one or more medication containers 130 contain at least one medication for the user. The set of programming instructions is executable by a processor.

In the exemplary embodiment, medication container monitor 122 communicates with medication containers 130 and database server 140 to detect when a medication container is touched by a user. For example, when a user picks up a medication container to consume a dosage of a medication, the user's unique fingerprint signature triggers a drug combination detection mechanism, via peer to peer communicator 134, between the medication containers 130 in the P2P network. The P2P network is maintained between the containers where the user's unique fingerprint signature was matched. For example, the user may arrange her medication containers 130 in the kitchen cupboard, together with medication containers that do not belong to the user. In this instance, peer to peer communicator 134 only detects the medication containers 130 that belong to user, and therefore only detects potential adverse combinations based on the user's P2P network.

Medication container monitor 122, in the exemplary embodiment, accesses the cloud based medication interaction API 116 to determine if the medication contained in the picked-up medication container will have an adverse reaction with any other medication consumed by the user recently, as denoted in medication consumption data 144, or expected to be consumed by the user in the near future, as depicted in medication database 142. For example, a patient may receive more than one medication prescription by more than one physician. Sometimes, one physician may not be aware of what another physician prescribes for the same patient. In such an instance, physician A may prescribe drug A, even though physician B has already prescribed drug B. In this example, drug A should never be combined with drug B due to clinically proven adverse reactions within a user's body. Unfortunately, physician A was not aware that physician B had prescribed drug B. In this instance, medication container monitor 122 serves as a verification check to detect adverse medication combinations for the patient. When the patient picks up the medication container containing drug B, medication container monitor 122 detects, via peer to peer communicator 134, that drug A was consumed by the patient two hours ago and determines, via medication interaction API, that drug B should not be consumed by the user until drug A is fully metabolized within the user's body, which takes five hours. However, medication container monitor 122 detects, while conducting an adverse reaction check on all peer medication containers 130 within the user's P2P network, that the user is scheduled to take drugB in 30 minutes. In this scenario, the medication container containing drug B may be automatically locked by medication assistant controller 118, for three more hours (until drug A is fully metabolized within the user's body), to protect the patient. In another embodiment, medication assistant controller 118 may notify the user and/or the user's physician via an alert, e-mail, phone call, text message, telephone call, or any other practical way to notify the patient that the current prescription combinations may be hazardous.

Medication consumption data receiver 124 includes a set of programming instructions in medication assistant controller 118, to receive from the one or more medication containers, medication consumption data of the user, wherein medication consumption data includes which medication of the at least one medication the user has taken, a time that the user took the at least one medication, and a quantity of the at least one medication taken by the user. The set of programming instructions is executable by a processor.

In an exemplary embodiment, when a medication container 130 is put back/closed, the amount of consumption of the drug by the user may be detected by various means. One such means may include computing the difference between the weights of the medication container 130 before it was opened and after it was closed. Another means may include visual recognition of the number of pills/tables/capsules within a medication container 130 before it was opened and after it was closed. In the case of a liquid medication, the amount of consumption by the user may be detected by measuring the amount of the liquid within the medication container 130 before it was opened and after it was closed.

In the exemplary embodiment, medication consumption data receiver 124 updates a patient's medication consumption history, both on the logic component of the medication container 130 and in medication consumption database 144, with the quantity of the medication consumed together with the timestamp of when the medication was consumed. Furthermore, medication assistant controller 124 broadcasts a message, via peer to peer communicator 134, to all of the other medication containers 130 within the user's P2P network, which is then used by those other medication containers 130 in individual adverse drug reaction checks.

Patient condition data receiver 126 includes a set of programming instructions in medication assistant controller 118, to receive patient condition data from one or more patient monitoring devices, in real-time, wherein the patient condition data comprises at least one measurement of a medical vital sign of the patient. The set of programming instructions is executable by a processor. In an exemplary embodiment, patient condition data may include, but is not limited to, one or more of blood pressure, pulse, cholesterol level, blood sugar level, heart rate, and body temperature of the user.

Patient condition data may be measured by external, transportable, or wearable, devices such as a heart rate monitor, glucometer, or FitBit for example. In one embodiment, the monitoring devices may convey patient condition data, in real-time, to patient condition data receiver 126. Medication assistant controller 118 may utilize received patient condition data to trigger a recommendation, an alert, or a notification to the user.

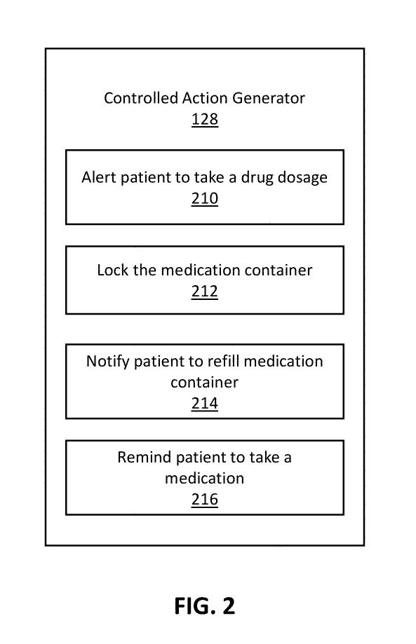

Referring now to FIGS. 1 and 2, controlled action generator 128 includes a set of programming instructions in medication assistant controller 118, to recommend that the patient consume a dosage amount of a medication at a designated time, from the one or more medication containers 130, based on the patient condition data (step 210). The set of programming instructions is executable by a processor. For example, if patient condition data receiver 126 detects that the user is experiencing a rapid heart rate and is scheduled to take 50 mg of drug A in five minutes, controlled action generator 128 may send an alert to the user, advising to only take 20 mg of drug A at the scheduled time since a lower dosage of drug A interacts more efficiently when a user is experiencing a rapid heart rate, as determined by medication interaction API 116. In the same vein, controlled action generator 128 may alert the user to take 30 mg of drug B to ease the user's rapid heart rate and bring the user's heart rate to a normalized rate, as determined by medication interaction API 116.

In another exemplary embodiment, and with continued reference to FIGS. 1 and 2, controlled action generator 128 may automatically lock one or more medication containers 130 if patient condition data receiver 124 indicates an adverse reaction or a potential adverse reaction to an already consumed medication or an about to be consumed medication from the one or more medication containers 130 (step 212). Since medication assistant controller 118 keeps, or has access to, a user's medication schedule, as well as a user's medication consumption history (e.g. <drugName, Q, timestamp>), controlled action generator 128 has the necessary information to preemptively lock a medication container associated with the adverse reaction or potential adverse reaction, to protect a user from consuming a medication that may be harmful, at a given time. Similarly, controlled action generator 128 may automatically lock one or more medication containers 130 associated with an overdose.

In other embodiments, if an adverse reaction, a potential adverse reaction, or an overdose is detected by medication assistant controller 118, controlled action generator 128 may additionally notify emergency services, a user's family member, and/or a user's physician. In the exemplary embodiment, medication assistant controller 118 may be capable of viewing a user's emergency medical record (EMR), which may contain contact information for the user's family member, or physician.

In another exemplary embodiment, and with continued reference to FIGS. 1 and 2, controlled action generator 128 may notify the user to refill one or more medication containers 130 with a particular medication (step 214). For example, if a user is going away on a vacation and will not be near a pharmacy to refill one or more medications, controlled action generator 128 may notify the user to refill one or more medications prior to the scheduled vacation. In the exemplary embodiment, controlled action generator 128 receives a user's electronic calendar information, from calendar 117, and determines based on the span of vacation days on calendar 117 whether an amount of a particular medication in the one or more medication containers 130 of user contains enough to last through the span of vacation days. For example, medication container monitor 122 may detect three pills remaining in medication container #1 today, while calendar 117 indicates that the user is going on vacation tomorrow for five days. Medication assistant controller 118 detects that the user's prescription calls for one pill, from medication container #1, to be taken on a daily basis. Controlled action generator 128 determines that medication container #1 will not have enough of a supply to last the entire vacation and, as such, outputs an alert to the user to refill medication container #1 prior to going away on vacation.

In another exemplary embodiment, and with continued reference to FIGS. 1 and 2, controlled action generator 128 may remind the user to consume a medication from the one or more medication containers 130, if medication consumption data of the user does not match the medication consumption instructions for the user (step 216). For example, a user's medication prescription may indicate to take drug A twice daily at 9:00 am and 9:00 pm. Medication consumption data receiver 124 may not receive a timestamp or detection of any removal of a dosage for drug A, which is contained in medication container #1, at 9:00 am. Nor did medication consumption data receiver 124 receive a timestamp, or a detection of any removal of a dosage of drug A from medication container #1, since yesterday's 9:00 pm timestamped dosage. As a result, controlled action generator 128 may send the user a reminder (e.g. e-mail, text message, push notification, for example) to take the dosage of drug A.

In the example embodiment, network 102 is a communication channel capable of transferring data between connected devices and may be a telecommunications network used to facilitate telephone calls between two or more parties comprising a landline network, a wireless network, a closed network, a satellite network, or any combination thereof. In another embodiment, network 102 may be the Internet, representing a worldwide collection of networks and gateways to support communications between devices connected to the Internet. In this other embodiment, network 102 may include, for example, wired, wireless, or fiber optic connections which may be implemented as an intranet network, a local area network (LAN), a wide area network (WAN), or any combination thereof. In further embodiments, network 102 may be a Bluetooth network, a WiFi network, or a combination thereof. In general, network 102 can be any combination of connections and protocols that will support communications between computing device 110, medication containers 130, and database server 140.

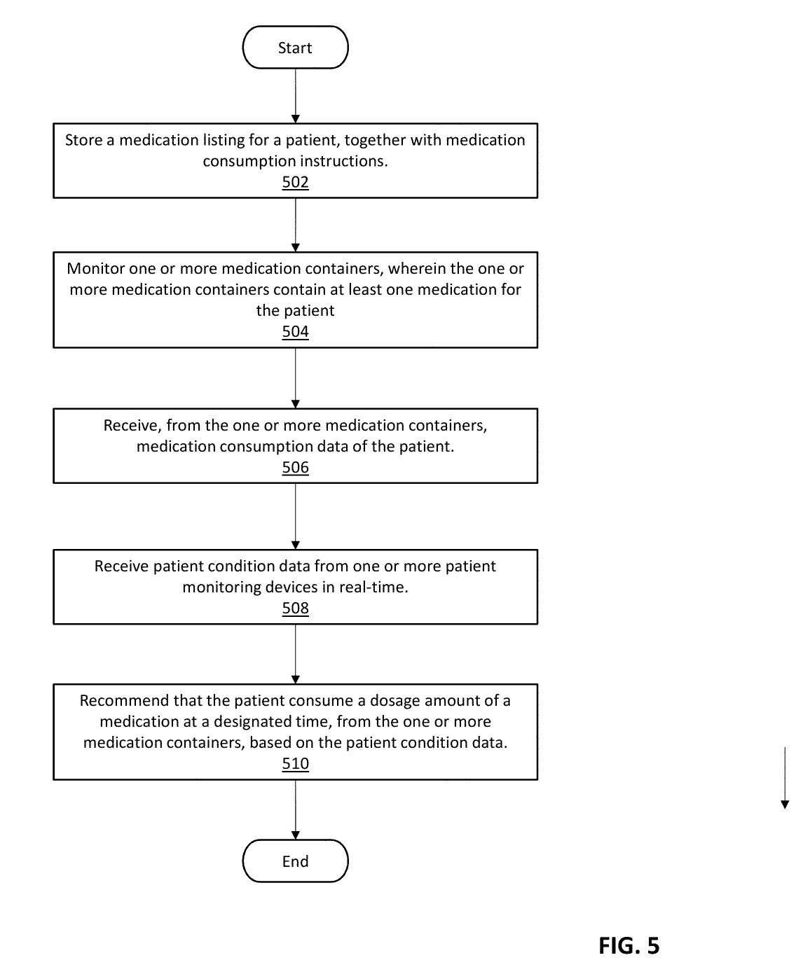

FIG. 5 is a flowchart depicting operational steps of a method for implementing a program that manages a device, according to an embodiment of the present invention. The methods of FIG. 5 are discussed further in reference to an illustrative example.

Referring now to FIGS. 1 and 5, medication assistant controller 118, via a processor, stores a medication listing for a patient, wherein the medication listing lists at least one medication for the patient, together with medication consumption instructions (step 502).

With continued reference to FIGS. 1 and 5, medication assistant controller 118, via a processor, monitors one or more medication containers, wherein the one or more medication containers contain at least one medication for the patient (step 504).

With continued reference to FIGS. 1 and 5, medication assistant controller 118, via a processor, receives, from the one or more medication containers, medication consumption data of the patient, wherein medication consumption data includes which medication of the at least one medication the patient has taken, a time that the patient took the at least one medication, and a quantity of the at least one medication taken by the patient (step 506).

In an exemplary embodiment, the one or more medication containers comprise multiple medication containers that exchange medication information and medication consumption data via peer-to-peer communication connections.

In an exemplary embodiment, the multiple medication containers 130 that exchange medication information and medication consumption data via peer-to-peer communication connections further includes triggering a drug combination analysis with one or more other medication containers, when a one or more initial medication container is lifted by the patient, wherein the patient is identified by a unique fingerprint, detecting the medication consumption data of the one or more initial medication containers, when the one or more initial medication containers is put back down by the patient, updating the medication consumption data with the dosage amount and the designated time of consumption of a medication consumed by the patient from the one or more initial medication containers, and broadcasting the medication consumption data from the one or more initial medication containers to the one or more other medication containers.

With continued reference to FIGS. 1 and 5, medication assistant controller 118, via a processor, receives patient condition data from one or more patient monitoring devices in real-time, wherein the patient condition data comprises at least one measurement of a medical vital sign of the patient (step 508). In exemplary embodiments, patient condition data includes any one or more of blood pressure, pulse, cholesterol level, blood sugar level, heart rate, and body temperature.

With continued reference to FIGS. 1 and 5, medication assistant controller 118, via a processor, recommends that the patient consume a dosage amount of a medication at a designated time, from the one or more medication containers, based on the patient condition data (step 510). In exemplary embodiments, recommendations may be calculated based on a patient's medical history, current drug regimen, age, and weight. In order for medication assistant controller 118 to recommend that the patient consume a dosage amount of a medication at a designated time, medication assistant controller 118 communicates with a cloud based medication interaction service comprising an API that monitors one or more medication reactions based on a quantity and a time of consumption of one or more medications, and determines that a recommended medication does not react adversely with the quantity and the time of consumption of the one or more medications that the patient has already consumed.

In an exemplary embodiment, medication assistant controller 118, via a processor, locks, automatically, the one or more medication containers 130 containing the medication associated with an adverse reaction or the potential adverse reaction, if the patient condition data indicates an adverse reaction or a potential adverse reaction to the medication from the one or more medication containers 130. In another embodiment, medication assistant controller 118, via a processor, locks, automatically, the one or more medication containers 130 containing the medication associated with an overdose, if the patient condition data indicates an overdose to the medication from the one or more medication containers 130.

In an exemplary embodiment, medication assistant controller 118, via a processor, reminds the patient to take a medication, if it is determined that the medication consumption data 144 does not match the medication consumption instructions.

In an exemplary embodiment, medication assistant controller 118, via a processor, receives electronic calendar information for the patient, wherein the electronic calendar information comprises event information, determines, based on the event information and the medication consumption instructions, whether an amount of medication in the one or more medication containers is enough to last through an event specified in the event information, and in response to determining that the amount of medication in the one or more medication containers is not enough to last through the event specified in the event information, outputting an alert to the patient indicating that at least one medication in the one or more medication containers should be refilled.

FIG. 6 is a block diagram depicting components of a computing device (such as computing device 110, medication containers 130, or database server 140, as shown in FIG. 1), in accordance with an embodiment of the present invention. It should be appreciated that FIG. 6 provides only an illustration of one implementation and does not imply any limitations with regard to the environments in which different embodiments may be implemented. Many modifications to the depicted environment may be made.

Computing device 110 may include one or more processors 902, one or more computer-readable RAMs 904, one or more computer-readable ROMs 906, one or more computer readable storage media 908, device drivers 912, read/write drive or interface 914, network adapter or interface 916, all interconnected over a communications fabric 918. Communications fabric 918 may be implemented with any architecture designed for passing data and/or control information between processors (such as microprocessors, communications and network processors, etc.), system memory, peripheral devices, and any other hardware components within a system.

One or more operating systems 910, and one or more application programs 911, such as medication assistant controller 118, may be stored on one or more of the computer readable storage media 908 for execution by one or more of the processors 902 via one or more of the respective RAMs 904 (which typically include cache memory). In the illustrated embodiment, each of the computer readable storage media 908 may be a magnetic disk storage device of an internal hard drive, CD-ROM, DVD, memory stick, magnetic tape, magnetic disk, optical disk, a semiconductor storage device such as RAM, ROM, EPROM, flash memory or any other computer-readable tangible storage device that can store a computer program and digital information.

Computing device 110 may also include a R/W drive or interface 914 to read from and write to one or more portable computer readable storage media 926. Application programs 911 on computing device 110 may be stored on one or more of the portable computer readable storage media 926, read via the respective R/W drive or interface 914 and loaded into the respective computer readable storage media 908.

Computing device 110 may also include a network adapter or interface 916, such as a TCP/IP adapter card or wireless communication adapter (such as a 4G wireless communication adapter using OFDMA technology). Application programs 911 on computing device 110 may be downloaded to the computing device from an external computer or external storage device via a network (for example, the Internet, a local area network or other wide area network or wireless network) and network adapter or interface 916. From the network adapter or interface 916, the programs may be loaded onto computer readable storage media 908. The network may comprise copper wires, optical fibers, wireless transmission, routers, firewalls, switches, gateway computers and/or edge servers.

Computing device 110 may also include a display screen 920, a keyboard or keypad 922, and a computer mouse or touchpad 924. Device drivers 912 interface to display screen 920 for imaging, to keyboard or keypad 922, to computer mouse or touchpad 924, and/or to display screen 920 for pressure sensing of alphanumeric character entry and user selections. The device drivers 912, R/W drive or interface 914 and network adapter or interface 916 may comprise hardware and software (stored on computer readable storage media 908 and/or ROM 906).

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

Referring now to FIG. 7, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 7 are intended to be illustrative only and that computing nodes 10 and cloud computing environment 50 can communicate with any type of computerized device over any type of network and/or network addressable connection (e.g., using a web browser).

Referring now to FIG. 8, a set of functional abstraction layers provided by cloud computing environment 50 (FIG. 7) is shown. It should be understood in advance that the components, layers, and functions shown in FIG. 8 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and controlling access to data objects 96.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Based on the foregoing, a computer system, method, and computer program product have been disclosed. However, numerous modifications and substitutions can be made without deviating from the scope of the present invention. Therefore, the present invention has been disclosed by way of example and not limitation.

1. A computer-implemented method that manages a device, comprising:
storing a medication listing for a patient, wherein the medication listing lists at least one medication for the patient with medication consumption instructions;
monitoring one or more medication containers, wherein the one or more medication containers comprise at least one medication for the patient;
receiving, from the one or more medication containers, medication consumption data of the patient, wherein the medication consumption data comprises which medication of the at least one medication the patient has taken, a time that the patient took the at least one medication, and a quantity of the at least one medication taken by the patient;
receiving patient condition data from one or more patient monitoring devices in real-time, wherein the patient condition data comprises at least one measurement of a medical vital sign of the patient; and
recommending that the patient consume a dosage amount of at least one medication at a designated time, from the one or more medication containers, based on the patient condition data.

2. The method of claim 1, further comprising:
in response to determining that the patient condition data indicates an adverse reaction or a potential adverse reaction to the medication from the one or more medication containers, locking, automatically, the one or more medication containers containing the medication associated with the adverse reaction or the potential adverse reaction; and
in response to determining that the patient condition data indicates an overdose, locking, automatically, the one or more medication containers containing the medication associated with the overdose.

3. The method of claim 1, further comprising:
alerting the patient to take a medication, if it is determined that the medication consumption data does not match the medication consumption instructions.

4. The method of claim 1, wherein the patient condition data includes any one or more of blood pressure, pulse, cholesterol level, blood sugar level, heart rate, and body temperature.

5. The method of claim 1, wherein recommending that the patient consume a medication, from the one or more medication containers based on the patient condition data, further comprises:
communicating with a cloud based medication interaction service comprising an application programming interface (API) that monitors one or more medication reactions based on a quantity and a time of consumption of one or more medications; and
determining that a recommended medication does not react adversely with the quantity and the time of consumption of the one or more medications consumed by the patient.

6. The method of claim 1, further comprising:
receiving electronic calendar information for the patient, wherein the electronic calendar information comprises event information;
determining, based on the event information and the medication consumption instructions, whether an amount of medication associated with the at least one medication in the one or more medication containers is enough to last through an event specified in the event information; and
in response to determining that the amount of medication associated with the at least one medication in the one or more medication containers is not enough to last through the event specified in the event information, outputting an alert to the patient to refill the at least one medication.

7. The method of claim 1, wherein the one or more medication containers comprise a plurality of medication containers that exchange medication information and medication consumption data via peer-to-peer communication connections.

8. The method of claim 7, further comprising:
triggering a drug combination analysis with one or more other medication containers, when a one or more initial medication containers is lifted by the patient,
wherein the patient is identified by a unique fingerprint;
detecting the medication consumption data of the one or more initial medication containers, when the one or more initial medication containers is put back down by the patient;
updating the medication consumption data with the dosage amount and the designated time of consumption of a medication consumed by the patient from the one or more initial medication containers; and
broadcasting the medication consumption data from the one or more initial medication containers to the one or more other medication containers.

9. A computer program product for implementing a program that manages a device, comprising a non-transitory tangible storage device having program code embodied therewith, the program code executable by a processor of a computer to perform a method, the method comprising:
storing, by the processor, a medication listing for a patient, wherein the medication listing lists at least one medication for the patient, together with medication consumption instructions;
monitoring, by the processor, one or more medication containers, wherein the one or more medication containers contain at least one medication for the patient;
receiving, by the processor, from the one or more medication containers, medication consumption data of the patient, wherein medication consumption data includes which medication of the at least one medication the patient has taken, a time that the patient took the at least one medication, and a quantity of the at least one medication taken by the patient;
receiving, by the processor, patient condition data from one or more patient monitoring devices in real-time, wherein the patient condition data comprises at least one measurement of a medical vital sign of the patient; and
recommending, by the processor, that the patient consume a dosage amount of a medication at a designated time, from the one or more medication containers, based on the patient condition data.

10. The computer program product of claim 9, further comprising:
if the patient condition data indicates an adverse reaction or a potential adverse reaction to the medication from the one or more medication containers, locking automatically, by the processor, the one or more medication containers containing the medication associated with the adverse reaction or the potential adverse reaction; or
if the patient condition data indicates an overdose, locking automatically, by the processor, the one or more medication containers containing the medication associated with the overdose.

11. The computer program product of claim 9, further comprising:
alerting, by the processor, the patient to take a medication, if it is determined that the medication consumption data does not match the medication consumption instructions.

12. The computer program product of claim 9, wherein recommending, by the processor, that the patient consume a medication, from the one or more medication containers based on the patient condition data, further comprises:
communicating, by the processor, with a cloud based medication interaction service comprising an API that monitors one or more medication reactions based on a quantity and a time of consumption of one or more medications; and
determining, by the processor, that a recommended medication does not react adversely with the quantity and the time of consumption of the one or more medications that the patient has already consumed.

13. The computer program product of claim 9, further comprising:
receiving, by the processor, electronic calendar information for the patient, wherein the electronic calendar information comprises event information;
determining, by the processor, based on the event information and the medication consumption instructions, whether an amount of medication in the one or more medication containers is enough to last through an event specified in the event information; and
in response to determining that the amount of medication in the one or more medication containers is not enough to last through the event specified in the event information, outputting, by the processor, an alert to the patient indicating that at least one medication in the one or more medication containers should be refilled.

14. The computer program product of claim 9, further comprising:
triggering, by the processor, a drug combination analysis with one or more other medication containers, when a one or more initial medication containers is lifted by the patient,
wherein the patient is identified by a unique fingerprint;
detecting, by the processor, the medication consumption data of the one or more initial medication containers, when the one or more initial medication containers is put back down by the patient;
updating, by the processor, the medication consumption data with the dosage amount and the designated time of consumption of a medication consumed by the patient from the one or more initial medication containers; and
broadcasting, by the processor, the medication consumption data from the one or more initial medication containers to the one or more other medication containers.

15. A computer system for implementing a program that manages a device, comprising:
one or more computer devices each having one or more processors and one or more tangible storage devices; and
a program embodied on at least one of the one or more storage devices, the program having a plurality of program instructions for execution by the one or more processors, the program instructions comprising instructions for:
storing, by the computer, a medication listing for a patient, wherein the medication listing lists at least one medication for the patient, together with medication consumption instructions;
monitoring, by the computer, one or more medication containers, wherein the one or more medication containers contain at least one medication for the patient;
receiving, by the computer, from the one or more medication containers, medication consumption data of the patient, wherein medication consumption data includes which medication of the at least one medication the patient has taken, a time that the patient took the at least one medication, and a quantity of the at least one medication taken by the patient;
receiving, by the computer, patient condition data from one or more patient monitoring devices in real-time, wherein the patient condition data comprises at least one measurement of a medical vital sign of the patient; and
recommending, by the computer, that the patient consume a dosage amount of a medication at a designated time, from the one or more medication containers, based on the patient condition data.

16. The computer system of claim 15, further comprising:
if the patient condition data indicates an adverse reaction or a potential adverse reaction to the medication from the one or more medication containers, locking automatically, by the computer, the one or more medication containers containing the medication associated with the adverse reaction or the potential adverse reaction; or
if the patient condition data indicates an overdose, locking automatically, by the computer, the one or more medication containers containing the medication associated with the overdose.

17. The computer system of claim 15, further comprising:
alerting, by the computer, the patient to take a medication, if it is determined that the medication consumption data does not match the medication consumption instructions.

18. The computer system of claim 15, wherein recommending, by the computer, that the patient consume a medication, from the one or more medication containers based on the patient condition data, further comprises:
communicating, by the computer, with a cloud based medication interaction service comprising an API that monitors one or more medication reactions based on a quantity and a time of consumption of one or more medications; and
determining, by the computer, that a recommended medication does not react adversely with the quantity and the time of consumption of the one or more medications that the patient has already consumed.

19. The computer system of claim 15, further comprising:
receiving, by the computer, electronic calendar information for the patient, wherein the electronic calendar information comprises event information;
determining, by the computer, based on the event information and the medication consumption instructions, whether an amount of medication in the one or more medication containers is enough to last through an event specified in the event information; and
in response to determining that the amount of medication in the one or more medication containers is not enough to last through the event specified in the event information, outputting, by the computer, an alert to the patient indicating that at least one medication in the one or more medication containers should be refilled.

20. The computer system of claim 15, further comprising:
triggering, by the computer, a drug combination analysis with one or more other medication containers, when a one or more initial medication containers is lifted by the patient,
wherein the patient is identified by a unique fingerprint;
detecting, by the computer, the medication consumption data of the one or more initial medication containers, when the one or more initial medication containers is put back down by the patient;
updating, by the computer, the medication consumption data with the dosage amount and the designated time of consumption of a medication consumed by the patient from the one or more initial medication containers; and
broadcasting, by the computer, the medication consumption data from the one or more initial medication containers to the one or more other medication containers.
